(19) United States
(12) Patent Application Publication
(10) Pub. No.: US 2015/0332584 A1
(43) Pub. Date: Nov. 19, 2015

(54) AUDIO SUBSYSTEM MONITORING MECHANISM FOR PATIENT MONITORS

(71) Applicant: General Electric Company, Schenectady, NY (US)

(72) Inventors: Jukka Mikael Tenkama, Helsinki (FI); Hannu Antero Pohjanpalo, Helsinki (FI); Miikka Lehikoinen, Vantaa (FI); Mika Rapo, Helsinki (FI); Tomi Markus Makela, Helsinki (FI); Jussi Ilmari Eronen, Helsinki (FI); Juha-Kyosti Hermanni Heleva, Helsinki (FI)

(73) Assignee: GENERAL ELECTRIC COMPANY, Schenectady, NY (US)

(21) Appl. No.: 14/686,081

(22) Filed: Apr. 14, 2015

Related U.S. Application Data
(60) Provisional application No. 61/994,426, filed on May 16, 2014.

(51) Int. Cl. G08B 29/12; A61B 5/00

(52) U.S. Cl. CPC G08B 29/126; A61B 5/7405

(57) ABSTRACT

A patient monitor that includes a system for testing the operation of a speaker included in the patient monitor is disclosed. The control unit of the patient monitor generates a low frequency test signal to the speaker. The speaker generates the test signal at a frequency that is below the normal audible range for a human ear. A current sensing device detects the current draw of the speaker during operation. The current draw is analyzed to determine whether the speaker is operating properly. A backup speaker is used to generate an audible alarm when the speaker is not working properly. A microphone can be included to detect the inaudible test signal.

### CROSS-REFERENCE TO RELATED APPLICATION

The present application is based on and claims priority to U.S. Provisional Patent Application Ser. No. 61/994,426 filed May 16. 2014, the disclosure of which is incorporated herein by reference.

### BACKGROUND

The present disclosure generally relates to a patient monitoring device. More specifically, the present disclosure relates to a system and method for continuously testing the operation of a speaker in a patient monitor when the patient monitor is in use.

Modern patient monitoring systems are able to monitor a wide variety of patient parameters from a patient and digitally display the monitored parameters on a display screen. In addition to displaying the monitor parameters, the patient monitor includes a control unit that compares the monitored patient parameters to alarm thresholds that can be either pre-set or user selectable. When the monitored patient parameter reaches or exceeds an alarm threshold, the patient monitor can generate a visual indication of an alarm condition and/or an audible alarm, In addition, audible alarms are created from other events related to patient conditions or the patient monitoring system. Since audible alarms can be heard by clinicians that are not currently viewing the patient monitor, audible alarms are vital components of patient monitoring systems.

Since audible alarms are vitally important in patient monitoring systems, it is desirable to have a system and method for testing, the audio system and the speaker contained within the patient monitor to make sure that the speaker is properly operated while at the same time not disturbing the patient being monitored.

### SUMMARY

The present disclosure generally relates to a system and method for testing the operation of a primary speaker of a patient monitor. More specifically, the present disclosure relates to a system and method for generating an inaudible test signal from the primary speaker and determining whether the test signal has been properly generated in the inaudible frequency range.

The system of the present disclosure includes a control unit that operates to generate a digital, low frequency inaudible test signal to an audio amplifier connected to the primary speaker. When the audio amplifier receives the low frequency test signal, the audio amplifier draws current from a power supply to drive the primary speaker. The primary speaker is driven to generate the inaudible test signal, which results in current draw from the power source.

In one embodiment, a current sensing device is positioned between the power source and the audio amplifier to detect the amount of current being drawn from the power source. The current sensing device provides a signal to an amplification circuit that, either indirectly or directly, is provided back to the control unit. If the current draw from the power supply is within a known parameter range, the control unit determines that the primary speaker is operating properly. However, if the current draw is outside the normal operating range, the control unit determines that the primary speaker is not operating correctly and the control unit generates alarm signals to either a backup speaker and/or a visual display.

In an alternate embodiment, the patient monitor can use a microphone that is positioned to detect the inaudible low frequency test signal generated by the primary speaker. Since the microphone can detect the inaudible low frequency test signals, the microphone provides this indication back to the control unit, which can determine whether the primary speaker is operating correctly.

In an alternate embodiment, the test signal can be measured directly as the voltage delivered to the speaker, or as the current driven into the speaker.

The inaudible low frequency test signal is generated by the control unit repeatedly during the operation of the patient monitor. The inaudible test signals do not disturb the patient or the operator and can be generated at either predetermined, repeatable intervals or on a random basis. However, it is contemplated that the test signals will be generated during the entire time of operation of the patient monitor to insure that the primary speaker is operating properly.

Various other features, objects and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIG. 1 is a view of a patient monitor configured to utilize the speaker testing system of the present disclosure;

FIG. 2 is a schematic illustration showing the communication between the control unit of the patient monitor and the testing system of the present disclosure;

FIG. 3 is a view similar to FIG. 2 with an alternate feedback to the control unit;

FIG. 4 is an alternate embodiment in which a microphone is used to further detect the operation of the primary speaker; and

FIG. 5 is a schematic flowchart showing the operation of the system to test the primary speaker and provide feedback to the control unit.

### DETAILED DESCRIPTION

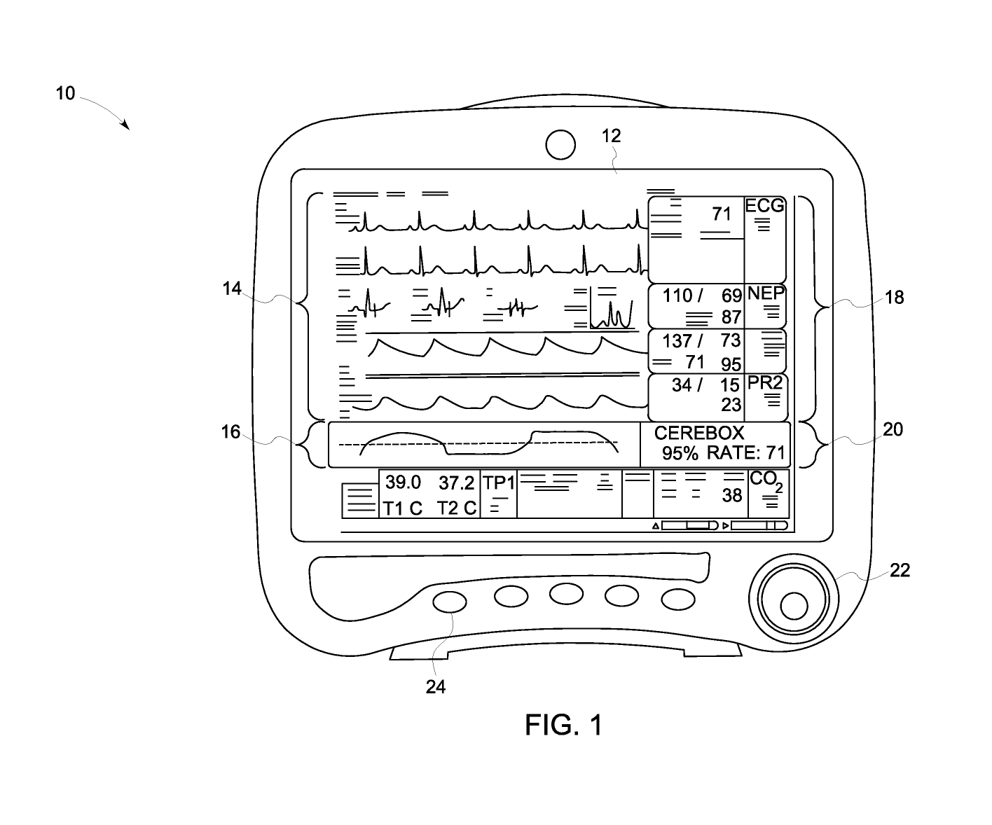

FIG. 1 illustrates a patient monitor 10 constructed in accordance with the present disclosure. The patient monitor 10 includes a visual display 12 that can be used to display a wide variety of parameters obtained from a patient. In the embodiment shown in FIG. 1, the patient monitor 10 includes multiple display areas that show waveforms 14, trending parameters over a series of time 16, current measured values 18, such as heart rate, blood pressure, and trending parameters as illustrated by reference numeral 20. Although various display sections are shown on the patient monitor 10, it should be understood that the patient monitor 1.0 could be configured in a variety of other ways depending upon the type of patient monitor being utilized, Additionally, the display 12 can be configured to show fewer or greater numbers of parameters depending, upon the patient monitoring conditions.

The patient monitor 10 includes a user input device 22 that allows a clinician to modify alarm parameters as well as control various other operating functions of the patient monitor. In the embodiment shown, the user input device 22 is a flexible knob that allows the user to visually move an indicator on the display 12. A series of user input buttons 24 can be included on the front face of the display. Alternatively, the display 12 can be configured as a touch screen such that the operator can enter and change parameters directly into the patient monitor through engagement with the touch screen.

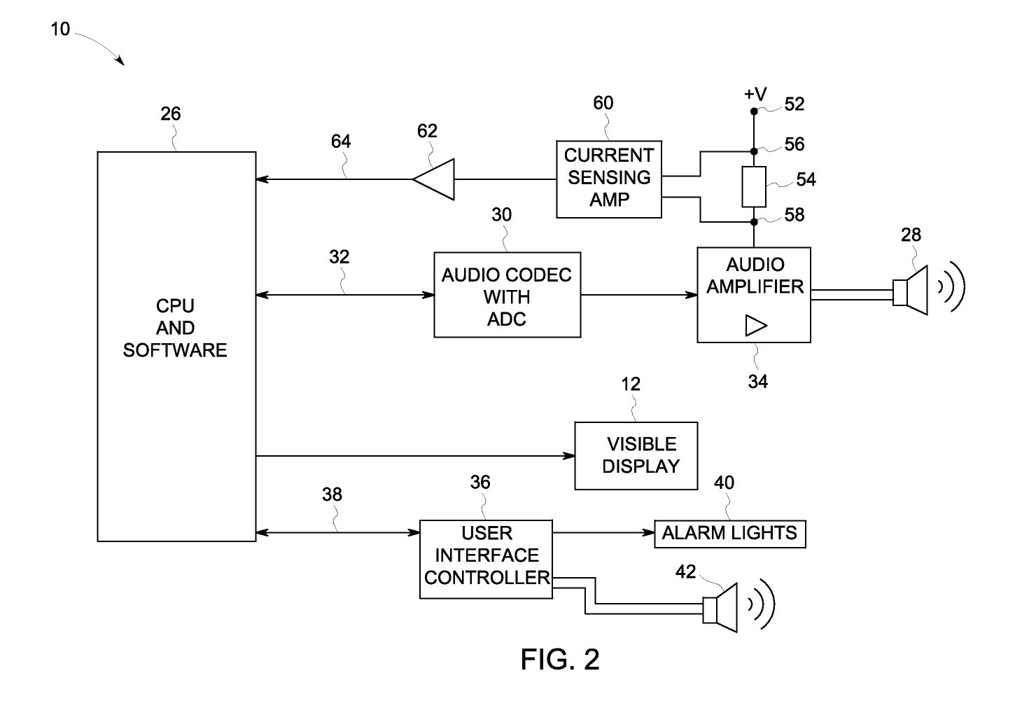

During operation, the patient monitor 10 receives a variety of patient parameters and compares the received patient parameters to alarm thresholds set and saved within the patient monitor. FIG. 2 illustrates the first embodiment of a portion of the internal operating components of the patient monitor 10, The patient monitor 10 includes a control unit 26 and software that operates to carry out a wide variety of functions with the patient monitor 10, including, generating audible and visual alarms, comparing monitored patient parameters to alarm limits and generally controlling the overall operation of the patient monitor 10. The embodiment shown in FIG. 1 illustrates the operating components of the patient monitor 10 that are needed to test the operation of the primary speaker 28 that is used to generate audible alarms.

As illustrated in FIG. 2, a control unit 26 communicates with an audio codec 30 through communication line 32, In the embodiment illustrated, the communication line 32 is a USB communication line that allows for bi-directional communication between the control unit 26 and the codec 30. The codec 30 is a device or a computer program that is capable of encoding and decoding a digital data stream or signal. In the embodiment shown, when the control unit 26 desires to generate an audible alarm, the control unit 26 generates a digital audio signal to the audio codec 30 along the USB communication line 32. Once this digital audio signal is received, the codec 30 converts the signal into an analog signal that is directed to an amplifier 34. The amplifier 34 amplifies the analog signal and provides the amplified, analog signal to the primary speaker 28.

In addition to the primary speaker 28, the patient monitor 1.0 further includes the display 12 that can receive both monitoring signals and visual alarm signals from the control unit 26. The patient monitor 10 further includes a user interface controller 36 that can communicate bi-directionally with the control unit 26 over a USB communication line 38. Through the communication line 38, the user interface controller 36 can communicate information to the control unit 26 and also receive commands and direction from the control unit 26. In the embodiment shown in FIG. 2, the user interface controller 36 is coupled to a series of alarm lights 40 as well as a backup speaker 42. In this manner, the control unit 26 is able to generate visual alarms utilizing both the alarm lights 40 as well as the visual display 12. The backup speaker 42 can be activated to generate an audible alarm if the primary speaker 28 is inoperable for any reason. The backup speaker 42 is designed to be utilized only when the primary speaker 28 is inoperable and thus does not need to be as dynamic as the primary speaker 28. In one embodiment of the disclosure, the backup speaker 28 can be a piezoelectric device that is both inexpensive and does not require the audio codec to operate.

In many situations, the visual alarms generated on the visible display 12 are not enough to alert a clinician if the audible alarm should fail. In such case, some urgent alarms can be left unnoticed and patient safety can be compromised if the primary speaker 28 is not functioning. The present disclosure provides a system and method for continuously testing the operation of the primary speaker 28 utilizing an inaudible test signal generated during the entire period of use of the patient monitor 10 to insure that the speaker 28 is properly operating. Since the test signal is inaudible, the test signal will not disturb the patient but will be effective in testing the operation of the primary speaker.

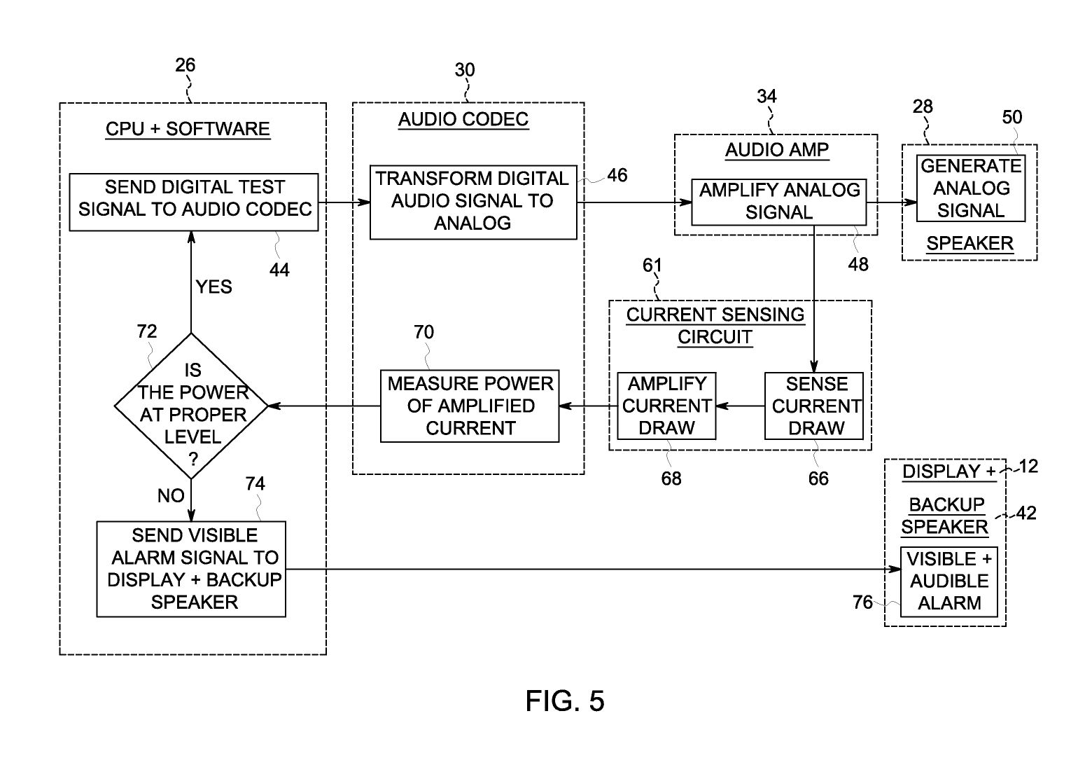

Referring now to FIGS. 2 and 5, the method and system for testing the operation of the primary speaker 28 will now be described. Initially, when the control unit 26 determines it is time to test the speaker 28, the control unit 26 sends a digital test signal to the audio codec 30, as illustrated by step 44. When the control unit 26 is generating an alarm, the digital audio signal sent to the audio codec 30 presents a digital representation of an audible signal that can be heard b the human ear. It is commonly understood that the normal range of human hearing is 20 Hz to 20 kHz with the human ear being most sensitive to frequencies between 2000 Hz and 5000 Hz.

In accordance with the testing system 10 of the present disclosure, when the control unit 26 wants to test the operation of the speaker, the control unit generates a digital test signal to the audio codec 30 that will have a frequency below the normal lowest frequency that can be heard by a human ear. The digital test signal sent from the control unit 26 to the audio codec 30 will be a short, low frequency pulse train that will be received by the audio codec 30 and transformed from the digital test signal to an analog, test signal, as shown b step 46. It is contemplated that the frequency of the analog test signal leaving the audio codec 30 will have a frequency below the normal hearing range of the human ear and will have fade-in lead and fade-out tail parts to guarantee that no harmonic frequencies will be heard. This analog test signal will be received by the amplifier 34, which will in turn amplify the analog test signal as illustrated in step 48. Once the analog, signal is amplified, the low frequency analog test signal will be provided to the primary speaker 28 which will generate the low frequency, inaudible analog signal as illustrated by step 50.

Referring back to FIG. 2, when the audio amplifier receives the low frequency test signal from the audio codec 30, the audio amplifier draws current from a power supply 52 through a current sensing device 54 to drive the speaker 28. In the most simplified version of the disclosure, the current sensing device 54 is a resistor that generates a voltage drop across the terminals 56, 58. The voltage drop across the terminals 56, 58 is provided to a current sensing amplifier 60. It is contemplated that alternate embodiments may include various other types of current sensing devices 54, such as a Hall sensor, a transformer having primary and secondary windings or a Rogowski coil that utilizes the flow of current from the power supply 52 to the audio amplifier 34 to induce a sensing, current. In each case, the current sensing device 54 detects the amount of current being drawn by the audio amplifier 34 to drive the speaker 28. If the primary speaker 28 is not connected to the audio amplifier 34 or for some reason is not operating, the audio amplifier 34 will either draw no current from the power supply 52 or will draw a very small amount of current from the power supply 52. In an alternate embodiment, the voltage delivered from the audio amplifier 34 to the speaker 28 or the current driven into the speaker 28 can be measured. This measured signal can be used in the same manner as the signal from the current sensing device 54.

The current sensing amplifier 60 passes the signal generated by the current sensing device 54 through a comparator 62 and provides a current sensing signal along the communication line 64 to the control unit 26. In the embodiment shown in FIG. 2, the control unit 26 can compare the signal on line 64 to a known, normal value that should be seen when the primary speaker 28 is operating properly. Thus, if the current signal on line 64 does not correspond to a normal value expected when the primary speaker 28 is operating properly, the control unit 26 determines that the primary speaker 28 is either nonfunctional or operating improperly. Based upon this determination, the control unit 26 can generate a signal to the backup speaker 42 and/or the alarm lights 40.

FIG. 5 illustrates the operational steps carried out in accordance with the present disclosure. As illustrated in Fig, 5, the current drawn from the power source is sensed at step 66 in the current sensing circuit 61, which may include the current sensing device 54, the current sensing amplifier 60, and the comparator 62. The sensed current is amplified in step 68 and provided to the audio codec 30 where the power of the current is measured at step 70 and provided hack to the control unit 26. In step 72, the control unit determines whether the power of the amplified current is at the proper level. If the power is at the proper level, which indicates that the primary speaker 28 is connected and operating properly, the system realms to step 44 and sends another digital test signal to the audio codec 30. It is contemplated that the control unit 26 can send the digital test signal at the low, inaudible frequency at a regular interval during operation of the patient monitor 10. As one illustrative example, it is contemplated that the low frequency pulse train will he sent from the CPU every twenty seconds during, operation of the patient monitor. Although twenty seconds is one contemplated interval between the pulse trains, it is contemplated that the interval between the low frequency pulse trains could be varied at either a longer or shorter interval. Additionally, the low frequency pulse trains could be sent at various different intervals during operation of the patient monitor 10 or at random times during operation. However, it is contemplated that it would be most desirable to continuously send the low frequency pulse trains at regular intervals during the operation of the patient monitor to ensure that the primary speaker 28 is properly operating at all times during the operation of the patient monitor 10.

As previously described, if the control unit 26 determines in step 72 that the current draw from the power supply is not at the proper level, the control unit proceeds to step 74 and sends visible and audible alarm signals to the display 12 and backup speaker 42 as illustrated in step 76. The signals are only sent to the backup speaker 42 and visible alarms sent to the display 12 when the system determines that the primary speaker 28 is not operating properly.

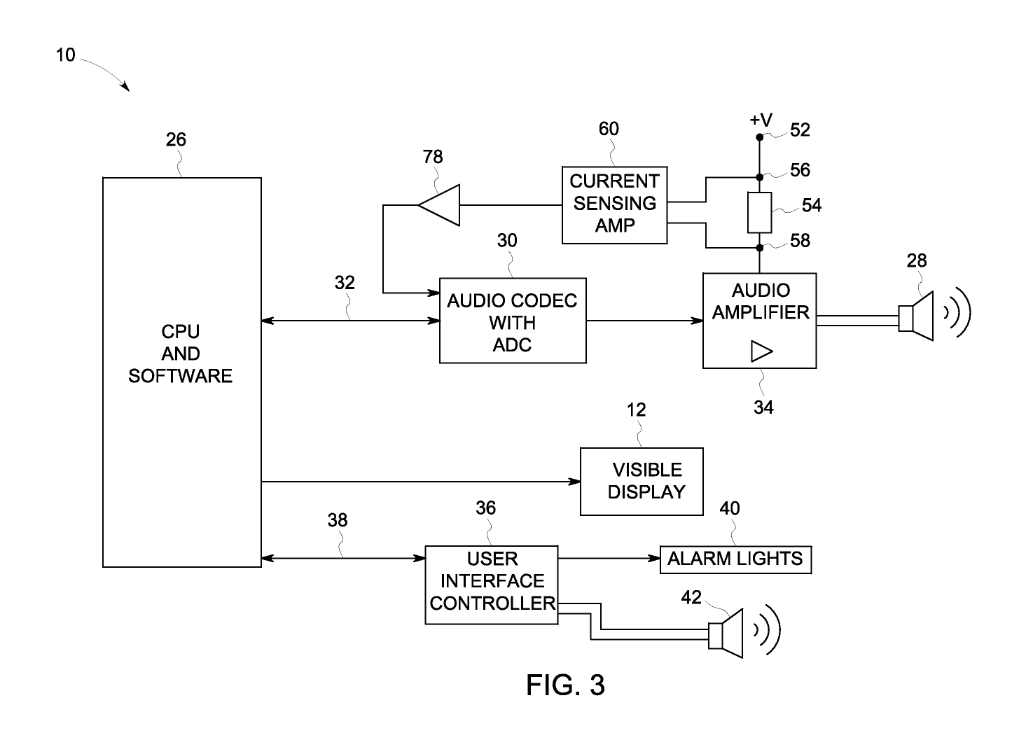

FIG. 3 illustrates an alternate embodiment of the present disclosure. The embodiment shown in FIG. 3 is similar to the embodiment of FIG. 2 and similar reference numerals are used to describe like components. In the embodiment shown in FIG. 3, the signal from the current sensing amplifier 60 is provided to a buffer 78 prior to being directed to the audio codec 30. The audio codec 30 is utilized to condition the signal from the buffer 78 before the signal is provided back to the control unit 26. In the embodiment of FIG. 3, the audio codec 30 further conditions the signal prior to providing the signal to the control unit 26. In this embodiment, some of the processing operations may be carried out by the audio codec 30 rather than the control unit 26, as was in the embodiment of FIG. 2. For example, the audio codec 30 may compare the power drawn by the audio amplifier 34 to a threshold level. The remaining components in operation remain consistent through the embodiments of FIGS. 2 and 3.

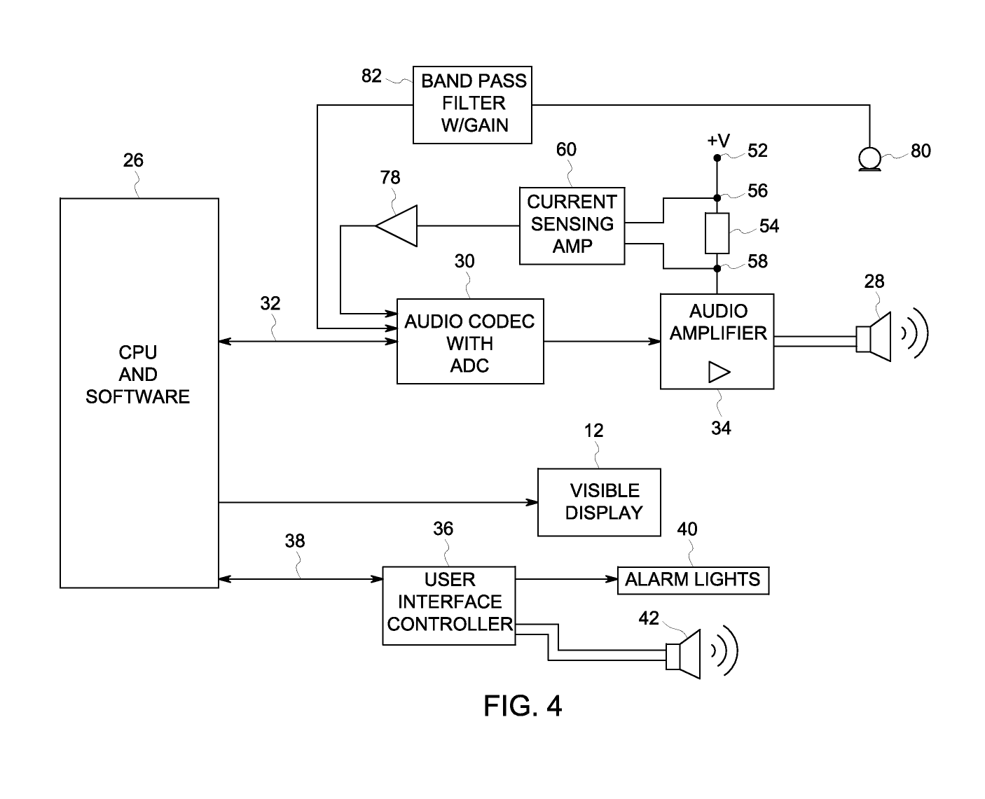

FIG. 4 illustrates a third alternate embodiment in accordance with the present disclosure. In the embodiment shown in FIG. 4, a microphone 80 is positioned to detect the output of the primary speaker 28. Since the system and method of the present disclosure utilizes a low frequency inaudible pulse train to test the operation of the primary speaker 28, the microphone 80 is designed to detect low frequency analog signals generated by the primary speaker 28 that cannot be detected by the human ear. The microphone 80 is connected to a band pass filter that includes gain as shown by reference numeral 82. The output from the band pass filter 82 is directed to the audio codec 30 for processing. However, it is also contemplated that the output of the band pass filter 82 could he provided directly to the control unit 26 depending upon the configuration of the system. In the embodiment shown in FIG. 4, the control unit can utilize both the output of the current sensing device 54 along with the signal from the microphone 80 to determine whether the primary speaker 28 is operating properly when the primary speaker 28 receives the low frequency pulse train from the control unit 26. The use of the microphone 80 provides yet another measurement for determining the proper operation of the primary speaker 28.

It is further contemplated that in the embodiment shown in FIG. 4, the current sensing, device 54 could be eliminated and only the microphone 80 utilized to determine the proper operation of the primary speaker 28 In such an embodiment, the primary speaker 28 would be utilized to generate the low frequency, inaudible test signal on a regular, continuous basis during operation of the patient monitor 10. However, it is believed that utilizing both the current sensing device 54 and the microphone 80 in combination would provide a more robust, failsafe system for detecting the low frequency, inaudible test signal from the primary speaker 28.

This written description uses examples to disclose the invention, including the best mode, and also to enable any person skilled in the art to make and use the invention. The patentable scope of the invention is defined by the claims, and may include other examples that occur to those skilled in the art Such other examples are intended to be within the scope of the claims if they have structural elements that do not differ from the literal language of the claims, or if they include equivalent structural elements with insubstantial differences from the literal languages of the claims.

We claim:

1. A method of testing operation of a speaker included in a patient monitor, comprising the steps of:
venerating a low frequency test signal in a control unit of the patient monitor;
operating the speaker of the patient monitor to generate the low frequency test signal, wherein the low frequency test signal is generated at a frequency below a normal audible range for a human ear;
determining a current draw of the speaker during the generation of the low frequency test signal; and
determining in the control unit whether the speaker is operating properly based on the current draw.

2. The method of claim 1 wherein the low frequency test signal is generated randomly during operation of the patient monitor.

3. The method of claim 1 wherein the low frequency test signal is generated at a periodic interval during operation of the patient monitor.

4. The method of claim 1 wherein the current draw is determined by a current sensing device positioned between a power supply and an audio amplifier driving the speaker.

5. The method of claim 4 wherein the current sensing device is one of a resistor, a Hall sensor, a transformer, and a Rogowski coil.

6. The method of claim 1 further comprising the step of generating an audible alarm signal to a backup speaker when the control unit determines that the speaker is not operating properly.

7. The method of claim 1 further comprising the step of positioning a microphone to detect the generation of the low frequency test signal by the speaker, wherein the control unit determines the proper operation of the speaker based on a signal from the microphone.

8. A patient monitoring system comprising:
a control unit;
a primary speaker coupled to the control unit;
an audio amplifier positioned between the control unit and the primary speaker to amplify a low frequency test signal generated by the control unit; and
a current sensing device positioned between the primary speaker and a power supply to determine a current draw of the speaker,
wherein the control unit determines the proper operation of the primary speaker based on the current draw.

9. The patient monitoring system of claim 8 wherein the low frequency test signal is below a normal audible frequency range for a human ear.

10. The patient monitoring system of 8 further comprising a microphone positioned to detect the low frequency test signal.

11. The patient monitoring system of claim 8 wherein the low frequency test signal is generated repeatedly during operation of the patient monitoring system.

12. The patient monitoring system of claim 11 wherein the low frequency test signal is generated at a periodic interval.

13. The patient monitoring system of claim 8 further comprising a backup speaker coupled to the control unit.

14. The patient monitoring system of claim 8 wherein the current sensing device is a resistor positioned between the power supply and the audio amplifier.

15. A method of testing the operation of a speaker in a patient monitor comprising the steps of:
operating the speaker of the patient monitor to generate a low frequency test signal, wherein the low frequency test signal is generated continuously during operation of the patient monitor and at a frequency below a normal audible range for a human ear;
detecting an operating parameter of the speaker during the generation of he low frequency test signal; and
determining whether the speaker is operating properly based on the detected parameter.

16. The method of claim 15 wherein the test signal is generated at periodic intervals.

17. The method of claim 15 wherein the test signal is generated randomly.

18. The method of claim 15 further comprising the step of generating an alarm when the speaker is not operating properly.

19. The method of claim 18 wherein the alarm is generated by a backup speaker.

20. The method of claim 15 wherein the operating parameter of the speaker is a voltage or current delivered to the speaker.
